(19) United States
(12) Patent Application Publication
(10) Pub. No.: US 2024/0423622 A1
(43) Pub. Date: Dec. 26, 2024

(54) SYSTEM AND METHOD FOR POSITIONING A MAGNETIC IMPLANT FOR FORMING AN ANASTOMOSIS AT A TARGET LOCATION IN THE DIGESTIVE TRACT

(71) Applicant: GT METABOLIC SOLUTIONS, INC., San Jose, CA (US)

(72) Inventors: Michel GAGNER, Montréal (CA); Todd A. KRINKE, Buffalo, MN (US); Thierry THAURE, San Jose, CA (US)

(73) Assignee: GT METABOLIC SOLUTIONS, INC., San Jose, CA (US)

(21) Appl. No.: 18/747,941

(22) Filed: Jun. 19, 2024

Related U.S. Application Data
(60) Provisional application No. 63/509,455, filed on Jun. 21, 2023.

(51) Int. Cl. A61B 17/11; A61B 17/00; A61B 90/00

(52) U.S. Cl. CPC A61B 17/1114; A61B 90/03; A61B 90/39; A61B 2017/00818; A61B 2017/00876; A61B 2017/1117; A61B 2090/033; A61B 2090/3966

(57) ABSTRACT

Systems and methods for positioning a magnetic implant for forming an anastomosis at a target location in a digestive tract of a patient are provided. The system can include first and second magnetic implants configured to magnetically couple to each other through two adjacent walls of the digestive tract at a desired site of the anastomosis to compress a portion of the two adjacent walls therebetween and form a necrotic area that becomes surrounded by a scarred edge following a healing time period. The system further can further include a positioning marker positionable at a target location corresponding to a first location on one side the desired site of the anastomosis for the first magnetic implant, the positioning marker being configured to intercept the first magnetic implant at the target location once swallowed by the patient.

### CROSS-REFERENCE TO RELATED APPLICATION

The present application claims priority from U.S. provisional patent application No. 63/509,455, filed on Jun. 21, 2023, entitled “SYSTEM AND METHOD FOR POSITIONING A MAGNETIC IMPLANT FOR FORMING AN ANASTOMOSIS AT A TARGET LOCATION IN THE DIGESTIVE TRACT”, the disclosure of which is hereby incorporated by reference in its entirety.

### TECHNICAL FIELD

The technical field generally relates to medical techniques for treating digestive tract conditions. In particular, the technical field relates to medical techniques for positioning a device for forming an anastomosis in the digestive tract.

### BACKGROUND

Metabolic surgeries and medical procedures to treat conditions associated with the digestive tract, diabetes and obesity often require alteration of the digestive tract through incisions, sutures, punctures and/or stapling, which can cause trauma to the organ being altered and lead to bleeding. For instance, bariatric surgery procedures can be used to treat obesity, and can be aimed at bypassing a portion of the stomach and/or the intestine. Such medical procedures can also lead to an increased risk of infection or other complications.

Magnetic compression anastomosis can be used in the context of medical procedures to treat conditions associated with the digestive tract. With magnetic compression anastomosis, necrosis is induced in tissue sandwiched between two magnets. A healing process takes place around the magnets, while the compressed tissue eventually dies and separates from surrounding living tissue. The magnets are released along with the necrotic tissue, leaving an open passage known as an anastomosis.

There remain a number of challenges with respect to surgery procedures in the digestive tract, particularly in the formation of an anastomosis.

### SUMMARY

In accordance with an aspect, there is provided a system for forming an anastomosis between two adjacent walls of a digestive tract of a patient, the system comprising:

first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; and a positioning marker positionable at a target location corresponding to a first location on one side the desired site of the anastomosis for the first magnetic implant, the positioning marker being configured to intercept the first magnetic implant at the target location once swallowed by the patient.

In some implementations, the positioning marker is engageable with a positioning marker guiding instrument.

In some implementations, the positioning marker comprises a positioning marker connector connectable to the positioning marker guiding instrument.

In some implementations, the positioning marker guiding instrument comprises a laparoscopic instrument.

In some implementations, the positioning marker guiding instrument comprises an endoscopic instrument.

In some implementations, the positioning marker comprises a floppy wire insertable through one of the two adjacent walls of the digestive tract.

In some implementations, the floppy wire comprises a spring coil.

In some implementations, the floppy wire comprises a distal segment that is magnetic, and a proximal segment that is non-magnetic.

In some implementations, the positioning marker comprises a pointed tip at a distal end thereof to facilitate passage through one of the two adjacent walls of the digestive tract.

In some implementations, the positioning marker comprises a needle at a distal end thereof to facilitate passage through one of the two adjacent walls of the digestive tract.

In some implementations, the positioning marker is configurable in a contracted configuration for placement at the target location and a deployed configuration for intercepting the first magnetic implant at the target location.

In some implementations, the positioning marker includes a deployable structure.

In some implementations, the deployable structure comprises a mesh.

In some implementations, the deployable structure comprises a T-fastener.

In some implementations, the deployable structure comprises a balloon.

In some implementations, the positioning marker further comprises a stabilizing feature to stabilize the positioning marker once at the target location.

In some implementations, the stabilizing feature comprises one or more of a clip and a hook.

In some implementations, the stabilizing feature comprises an endoscopic suture.

In some implementations, the positioning marker comprises a defeatable portion.

In some implementations, the defeatable portion is defeatable mechanically using an endoscope.

In some implementations, the defeatable portion is defeatable via a dissolution mechanism or a degradation mechanism.

In some implementations, the positioning marker comprises a positioning marker magnet having an opposite magnetic pole relative to a magnetic pole of the first magnetic implant such that the first magnetic implant is attracted to the magnet of the positioning marker.

In some implementations, the positioning marker comprises a laparoscopic clip.

In some implementations, the laparoscopic clip comprises a positioning marker magnet having an opposite magnetic pole relative to a magnetic pole of the first magnetic implant such that the first magnetic implant is attracted to the magnet of the positioning marker.

In some implementations, the positioning marker comprises an endoscopic clip.

In some implementations, the endoscopic clip comprises a positioning marker magnet having an opposite magnetic pole relative to a magnetic pole of the first magnetic implant such that the first magnetic implant is attracted to the magnet of the positioning marker.

In accordance with another aspect, there is provided a system for forming an anastomosis between two adjacent walls of a digestive tract of a patient, the system comprising:
first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; and
wherein at least one of the first and second magnetic implants comprises a fluorescent marker or radiopaque marker to detect a positioning of the at least one of the first and second magnetic implants as the at least one of the first and second magnetic implants travels through the digestive tract once swallowed by the patient.

In some implementations, the at least one of the first and second magnetic implants comprises a housing configured to house a magnet therein, and the fluorescent marker or the radiopaque marker is incorporated into the housing.

In some implementations, the fluorescent marker comprises one or more of indocyanine green, CH1055 or a fluorescein.

In accordance with another aspect, there is provided a method for positioning at least one of first and second magnetic implants configured for forming an anastomosis between two adjacent walls of a digestive tract of a patient, the method comprising:
inserting the first magnetic implant into the digestive tract via a mouth of the patient so that the first magnetic implant travels through the digestive tract;
positioning a positioning marker at a target location corresponding to a first location on one side of a desired site of the anastomosis for the first magnetic implant;
intercepting the first magnetic implant on the one side of the desired site of the anastomosis via the positioning marker;
delivering the second magnetic implant to a second location on another side of the desired anastomose site;
magnetically coupling the first and second magnetic implants to each other through the two adjacent vessel walls of the digestive tract to compress a portion of the two adjacent walls therebetween and form a necrotic area during a healing time period.

In some implementations, the first magnetic implant reaches the target location naturally due to peristalsis.

In some implementations, delivering the second magnetic implant to the second location comprises releasably engaging the second magnetic implant with a delivery catheter insertable in a working channel of an endoscope.

In some implementations, the method further comprises engaging the positioning marker with a positioning marker guiding instrument.

In some implementations, the positioning marker guiding instrument comprises a laparoscopic instrument.

In some implementations, positioning the positioning marker at the target location comprises introducing the laparoscopic instrument into an abdominal cavity of the patient.

In some implementations, positioning the positioning marker at the target location corresponding to the first location on the one side of a desired site of the anastomosis further comprises introducing at least a portion of the positioning marker into the digestive tract.

In some implementations, intercepting the first magnetic implant on the one side of the desired site of the anastomosis via the positioning marker comprises physically intercepting the first magnetic implant.

In some implementations, intercepting the first magnetic implant on the one side of the desired site of the anastomosis comprises magnetically coupling a positioning marker magnet of the positioning marker and the first magnetic implant.

In some implementations, positioning the positioning marker at the target location corresponding to the first location on the one side of a desired site of the anastomosis comprises placing a laparoscopic clip as the positioning marker at the target location.

In some implementations, intercepting the first magnetic implant on the one side of the desired site of the anastomosis via the positioning marker comprises physically intercepting the first magnetic implant.

In some implementations, the laparoscopic clip is a magnetic laparoscopic clip.

In some implementations, intercepting the first magnetic implant on the one side of the desired site of the anastomosis comprises magnetically coupling the magnetic laparoscopic clip and the first magnetic implant.

In some implementations, the positioning marker guiding instrument comprises an endoscopic instrument.

In some implementations, positioning the positioning marker at the target location corresponding to the first location on the one side of a desired site of the anastomosis comprises placing an endoscopic clip at the target location.

In some implementations, intercepting the first magnetic implant on the one side of the desired site of the anastomosis via the positioning marker comprises physically intercepting the first magnetic implant.

In some implementations, the endoscopic clip is a magnetic endoscopic clip.

The method of claim 46, wherein intercepting the first magnetic implant on the one side of the desired site of the anastomosis comprises magnetically coupling the magnetic endoscopic clip and the first magnetic implant.

In accordance with another aspect, there is provided a method for positioning at least one of first and second magnetic implants configured for forming an anastomosis between two adjacent walls of a digestive tract of a patient, the method comprising:
inserting the first magnetic implant into the digestive tract via a mouth of the patient so that the first magnetic implant travels through the digestive tract, the first magnetic implant comprising a fluorescent marker or radiopaque marker;
detecting a positioning of the at least one of the first and second magnetic implants as the at least one of the first and second magnetic implants travels through the digestive tract once swallowed by the patient based on the fluorescent marker or radiopaque marker;
delivering the second magnetic implant to a second location on another side of the desired anastomose site;
magnetically coupling the first and second magnetic implants to each other through the two adjacent vessel walls of the digestive tract to compress a portion of the two adjacent walls therebetween and form a necrotic area during a healing time period.

In some implementations, the first magnetic implant travels down the digestive tract naturally due to peristalsis.

In some implementations, delivering the second magnetic implant to the second location comprises releasably engaging the second magnetic implant with a delivery catheter insertable in a working channel of an endoscope.

In some implementations, the method further comprises inserting a laparoscopic instrument in the abdominal cavity of the patient to position the at least one of the first and second magnetic implants at a target location corresponding to a first location on one side the desired site of the anastomosis.

In accordance with another aspect, there is provided a system for forming an anastomosis between two adjacent walls of a digestive tract of a patient, the system comprising:
first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; and
a positioning marker comprising an umbilicus engaging portion positionable externally into an umbilicus of the patient to intercept one of the first and second magnetic implants at a target location once the magnetic implant is swallowed by the patient by magnetically coupling with the one of the first and second magnetic implants.

In some implementations, the one of the first and second magnetic implants is the first magnetic implant, and the target location corresponds to a first location on one side the desired site of the anastomosis for the first magnetic implant.

In some implementations, the positioning marker further comprises an abdominal wall abutting portion configured to be placed onto an abdominal wall of the patient to stabilize the umbilicus engaging portion into the umbilicus.

In some implementations, the positioning marker further comprises a belt, the positioning marker being engaged with the belt and the belt being configured to further stabilize the positioning marker.

### BRIEF DESCRIPTION OF THE DRAWINGS

The attached figures illustrate various features, aspects and implementations of the technology described herein.

FIG. 1 is an exploded perspective view of a first magnetic implant shown on one side of a desired site of an anastomosis and of a second magnetic implant shown on another side of the desired site of the anastomosis, with a vessel wall of a first hollow organ and a vessel wall of a second hollow organ being shown therebetween, in accordance with an implementation.

FIG. 2 is a perspective view of the first and second magnetic implants shown in FIG. 1, with the first magnetic implant being shown in contact with the vessel wall of the first hollow organ and the second magnetic implant being shown in contact with the vessel wall of the second hollow organ, at the desired site of the anastomosis.

FIG. 3 is a side view of the first and second magnetic implants shown in FIG. 2.

FIG. 4A is a perspective view of a magnetic implant and of a retention member, in accordance with an implementation.

FIG. 4B is a side view of the magnetic implant of FIG. 4A.

FIG. 5 is an exploded view of the magnetic implant of FIG. 4A.

FIG. 6 is a cross-sectional view of the magnetic implant of FIG. 4A.

FIG. 7 is a perspective view of first and second magnetic implants having an annular shape, in accordance with an implementation.

FIG. 8 is a side view of a positioning marker inserted into an abdominal cavity of a patient and a lumen of a hollow organ via a trocar that is placed through the abdominal wall.

FIG. 9 is a side view of the positioning marker of FIG. 8, the positioning marker being inserted into the lumen of the hollow organ and being positioned to intercept a magnetic implant traveling in the hollow organ.

FIG. 10A is a cross-sectional side view of an abdominal cavity of a patient and a perspective view of a hollow needle for inserting a positioning marker into a hollow organ.

FIG. 10B is a cross-sectional view of the abdominal cavity of the patient and a perspective view of the hollow needle shown in FIG. 10A, the hollow needle being retrieved from the hollow organ, a positioning marker configured as a T-fastener being inserted into the hollow organ via the hollow needle, the positioning marker including a distal end that rotates to switch the positioning marker from a contracted configuration to a deployed configuration.

FIG. 10C is a cross-sectional view of the abdominal cavity of the patient and a perspective view of the positioning marker of FIG. 10B, shown in a deployed configuration with the distal end being abutted against a luminal wall surface of the hollow organ.

FIG. 11 is a cross-sectional view of an abdominal cavity of a patient, with a portion of a positioning marker received into the umbilicus and a magnetic implant located in the small intestine that is magnetically attracted to the positioning marker.

FIG. 12 is a top view of the positioning marker of FIG. 11, the positioning marker being combined with a belt.

FIG. 13 is a schematic side view of a positioning marker engaged with a wall of a hollow organ, in accordance with an implementation.

FIG. 14 is a schematic side view of a positioning marker engaged with a wall of a hollow organ, in accordance with another implementation.

FIG. 15 is a schematic side view of a positioning marker engaged with a wall of a hollow organ, in accordance with another implementation.

FIG. 16 is a perspective view of a magnetic implant and a positioning marker incorporated into the housing of the magnetic implant, in accordance with an implementation.

### DETAILED DESCRIPTION

Techniques described herein relate to systems, devices and methods for assisting in the deployment and coupling of magnetic implants used for forming a magnetic compression anastomosis between two adjacent walls of hollow organs of the digestive tract of a patient, in the context of procedures to treat various medical conditions associated with the digestive tract. Such assistance can include positioning a positioning marker at a target location corresponding to a first location on one side of a desired site of the anastomosis for the first magnetic implant. Another positioning market can also optionally be positioned at another target location corresponding to a second location on another side of the desired site of the anastomosis for the second magnetic implant.

Then, the first and second magnetic implants can be magnetically coupled together to compress the tissue of the adjacent walls therebetween. Compression of the wall tissue between the two magnetic implants results in a necrotic area that corresponds approximately to the surface area of the compression surface of the pair of magnetic implants. Over time, the necrotic area becomes surrounded by an edge of scar tissue, or scarred edge. The formation of scar tissue can include collagen fiber deposition, neovascularization, and epithelial regeneration, and represents a dynamic equilibrium involving cells, their milieu, and the extracellular matrix. Cytokines secreted by platelets and inflammatory cells can promote the formation of new blood vessels and collagen synthesis which, in dynamic balance with collagen degradation, can contribute to determining the healing response. Two components of collagen are hydroxyproline and hydroxylysine, with hydroxyproline being synthesized under conditions of oxidative stress via the hydroxylation of proline, and being involved in the cellular transport of collagen. The synthesis and transport of wound collagen can thus be understood by monitoring the hydroxyproline content of the wound. The edge of scar tissue can thus be characterized by the fusion, or mechanical bonding, of the walls of each hollow organ through which the anastomosis is formed that occurs in part via fibrosis mechanisms. The scarred edge can thus form a fluid-tight seal around the anastomosis.

Various implementations and features of the magnetic implants and associated positioning makers will now be described in greater detail in the following paragraphs.

### General Description of the System for Forming an Anastomosis

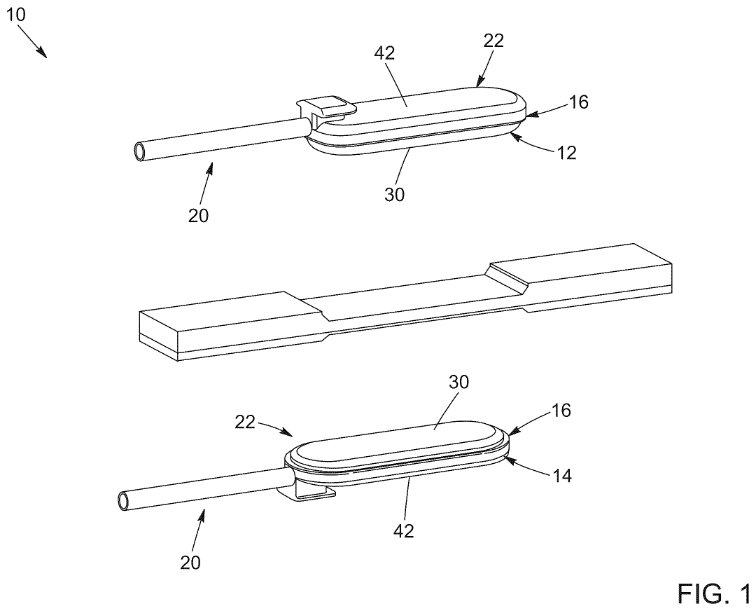

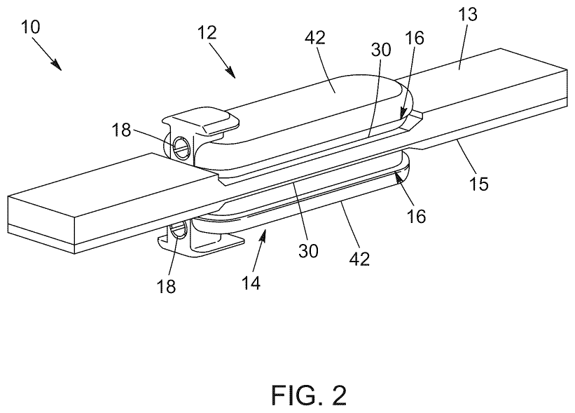

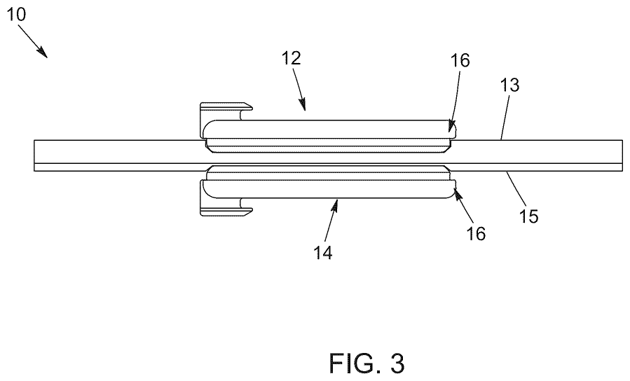

With reference to FIGS. 1 to 6, a system 10 for forming an anastomosis between two adjacent walls of hollow organs of the digestive tract is shown. Referring more particularly to FIG. 1, in the implementation shown, the system 10 includes a first magnetic implant 12 for implantation at a first location in the digestive tract, for instance in the stomach, and a second magnetic implant 14 for implantation at a second location in the digestive tract, for instance in the jejunum. It is to be understood that the term “implant” as used herein refers to a device that is implanted in the digestive tract for a certain period of time, e.g., the healing time period, and that it can be used interchangeably with the term “device” or “component” for instance. In this implementation, the stomach represents a first hollow organ of the digestive tract into which the first magnetic implant 12 can be implanted, and the jejunum represents a second hollow organ into which the second magnetic implant 14 can be implanted, so as to compress a portion of the stomach wall 13 and a portion of the jejunum wall 15 therebetween. It is to be understood that other hollow organs can receive the first and second magnetic implants in accordance with a treatment plan determined by a healthcare provider.

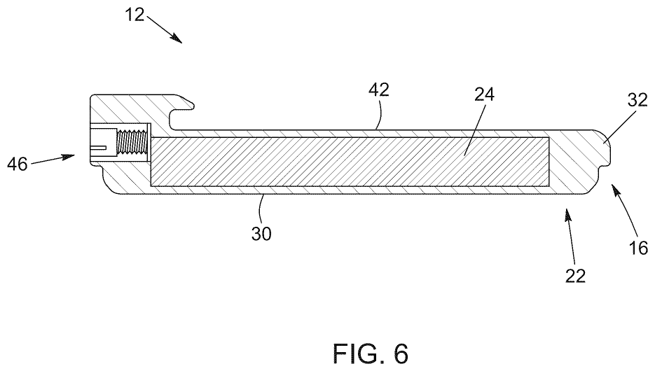

In FIGS. 1 to 6, each one of the first magnetic implant 12 and the second magnetic implant 14 is associated with a retention member 16, which is illustrated as corresponding to a flange 32 in FIG. 6. In the implementation shown, each one of the first magnetic implant 12 and the second magnetic implant 14 also includes a connecting member 18 that can be releasably engageable with a connector 20, which in FIG. 1 is identified as a delivery catheter. In other words, the magnetic implant 12, 14 can include a feature that enables its connection to a connector 20 for navigating the magnetic implant 12, 14 to a desired site for creating the anastomosis. In turn, the connecting member 18 can include any feature that enables a releasable connection of the magnetic implant 12, 14 with the connector 20. In FIGS. 1 to 6, the connecting member 18 is shown as a catheter attachment that includes a catheter attachment assembly 46. The catheter attachment is configured as a receiving cavity that can receive a distal end of the connector 20 therein, which as mentioned above can be a delivery catheter.

In some implementations and as shown in FIGS. 1 to 6, the magnetic implant 12, 14 can include a housing 22 that encloses a magnet 24 therein. The housing 22 can include for instance an outward portion 26 and an inward portion 28. The inward portion 28 includes the portion of the housing that faces the corresponding other magnetic implant and is involved in the magnetic compression of the tissue, while the outward portion 26 is on the opposed side of the magnetic implant facing away from the tissue being compressed. In this example, the two housing components surround the magnet and can be coupled together around a periphery thereof. Other housing constructions are also possible, where one or more housing components are used to partly or fully enclose the magnet.

The positioning of at least one of the magnetic implants at the desired site of the anastomosis can be performed via the assistance of a positioning maker interacting with the at least one of the magnetic implants within the digestive tract of the patient, the positioning marker having previously been positioned at a target location on one side of a desired site of the anastomosis. In some implementations, the positioning maker can be introduced in the digestive tract of the patient by a healthcare provider, such as a physician, using a minimally invasive surgery, and placed at the target location. Minimally invasive surgeries can include laparoscopic surgeries, which typically includes cooperation of a laparoscopic instrument with a trocar to facilitate introduction of the laparoscopic instrument into the abdominal cavity, percutaneous laparoscopy, which can enable introduction into the abdominal cavity without the use of a trocar, and Natural Orifice Transluminal Endoscopic Surgery (NOTES) procedures, for example.

Each of these components of the system for forming an anastomosis will now be described in further detail.

### Description of the Magnetic Implants

Still referring to FIGS. 1 to 6, the first magnetic implant 12 is a device that is implantable into a first hollow organ of the digestive tract of a patient at a site of a desired anastomosis via the lumen of the first hollow organ. Examples of hollow organs of the digestive tract include the oesophagus, stomach, duodenum, jejunum, ileum, colon, biliary tract, and pancreatic duct. A site of desired anastomosis can be determined according to the condition of the patient, and this aspect will not be discussed further in the context of the present description. As used herein, the expression “magnetic implant” refers to a structure that can be implanted into the chosen hollow organ of the digestive tract, and that can be magnetically attracted to another magnetic implant due to magnetic forces. In some implementations, the magnetic implant can consist of a magnet. In some implementations, the magnetic implant can include a magnet and one or more additional features, such as a housing and/or a connecting member. The two magnetic implants can be substantially the same as each other, or different, in terms of their shape, configuration, construction, and/or material make-up.

The first magnetic implant 12 is used with a second magnetic implant 14 to form a pair of magnetic implants. The second magnetic implant 14 is a device implantable into a second hollow organ of the digestive tract of the patient to the site of the desired anastomosis via the lumen of the second hollow organ. The second hollow organ of the digestive tract is located in sufficiently close proximity of the first hollow organ to enable the convergence of the respective wall tissue of the first hollow organ and the second hollow organ to eventually form the anastomosis.

The first and second magnetic implants 12, 14 are configured to remain within the digestive tract for at least a given healing time period. The healing time period enables ischemic pressure necrosis of the anastomosis area while providing enough time for the edge of scar tissue to form. In some implementations, after approximately 3 to 5 days following implantation of the pair of magnetic implants at the desired site of the anastomosis, the periphery of the anastomosis is strengthened by collagen deposition, with the formation an edge of scar tissue having an increased tensile strength occurring at an estimated of approximately 7 to 10 days following implantation. The duration for forming the scar tissue can vary depending on the overall health of the individual patient, and depending on the specific parts of the digestive tract being joined. The scar tissue can also gain strength over the course of several additional weeks. In some implementations, it may be desirable for the magnetic implants to be released and passed out of the body of the patient about two weeks after implantation. In some implementations, the healing time period can be about two weeks, or at least two weeks.

Each one of the first and second magnetic implants 12, 14 can have any suitable shape and size determined in accordance with their intended purpose. In some implementations, the size and the shape of the magnetic implant can be determined for instance in accordance with the characteristics of the site of the desired anastomosis, the delivery technique chosen to deliver the magnetic implant to the site of the desired anastomosis, and so on. In some implementations, the magnetic implant can have for example an elliptic shape, a circular shape, an elongated shape, a rectangular shape, an octagonal shape, or any other polygonal shape in terms of its cross-section. The magnetic implant can include rounded corners to facilitate navigation into the digestive tract. The magnetic implant can have an aspect ratio of about 1:1 (e.g., in the case of a circular cross-section) or an aspect ratio of about 1:2 to 1:40, about 1:3 to 1:20, about 1:4 to 1:15, for example, or another aspect ratio. In some implementations, the shape and size of the retention member 16 can be adapted in accordance with the shape and size of the corresponding magnetic implant. For instance, in some implementations, the height of the magnetic implant can be proportional to the thickness of the magnet contained therein and hence desired magnetic strength.

Each of the first and second magnetic implants 12, 14 includes a compression surface 30 that is configured to contact the tissue of the corresponding hollow organ. The compression surface 30 can also be referred to as a tissue-contacting surface, since it is the surface of the magnetic implant that is eventually in contact with the interior wall of the hollow organ once the magnetic implant is delivered to the site of the desired anastomosis. Each of the first and second magnetic implants 12, 14 also includes a lumen-oriented surface 42 opposite the tissue-contacting surface, the lumen-oriented surface generally facing the lumen of the hollow organ once the magnetic implant is delivered to the site of the desired anastomosis.

In some implementations, the compression surface 30 can be substantially continuous and flat, as shown in FIGS. 1 to 6. This can contribute to evenly distribute the force of the magnetic implant onto the tissue once the first and second magnetic implants 12, 14 are magnetically coupled together. In other implementations, the compression surface 30 of the first magnetic implant 12 can have a complementary shape compared to the compression surface 30 of the second magnetic implant 14. For instance, the first magnetic implant 12 can have a curvilinear surface that is inwardly curved, i.e., concave, and the second magnetic implant 14 can have a complimentary curvilinear surface that is outwardly curved, i.e., convex, for the first magnetic implant 12 to mate therewith.

In other implementations, the compression surface 30 can include features such as ridges, crests, furrows, grooves, and the like. For instance, the compression surface 30 of the first magnetic implant 12 can include a series of ridges, and the second magnetic implant 14 can include a complimentary series of furrows such that when the first and second magnetic implants 12, 14 are magnetically coupled, the first and second magnetic implants 12, 14 can interlock and/or self-align to increase the stability of their positioning on their respective sides of the first and second hollow organs.

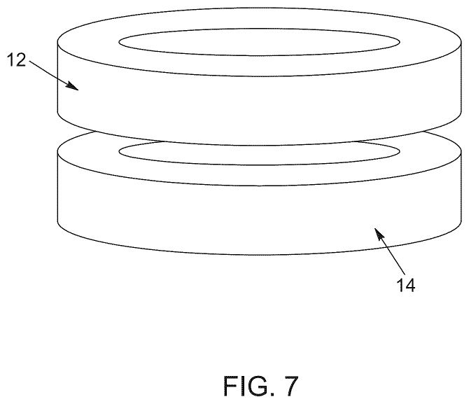

In yet other implementations, the compression surface 30 can be discontinuous and include void portions, i.e., where the wall tissue is not contacted by a portion of the magnetic implant. For instance, with reference to FIG. 7, the first and second magnetic implants 12, 14 and/or the compression surfaces 30 thereof can have a donut shape, or annular shape. In some implementations, the first and second magnetic implants 12, 14 can have a similar size and a similar or complimentary shape to facilitate the magnetic coupling through the wall tissues of the hollow organs. In other implementations, the first and second magnetic implants 12, 14 can have a different size and shape depending on the application and the sought-after characteristics of the resulting anastomosis.

In some implementations, the magnetic implant 12, 14 can include one or more magnets. The magnet 24 can be any type of suitable magnet composed of the appropriate material. In some implementations, the magnet 24 can be chosen according to its attractive force, i.e., according to the pressure that will be exerted on the surface area of the tissue that will eventually be compressed between the first and second magnetic implants 12, 14. Factors influencing the attractive force of the magnet 24 can include the shape of the magnet 24, the thickness of the magnet 24, the material of which the magnet 24 is made, etc. Example materials include neodymium magnets (e.g., NdFeB magnets), rare earth magnets, and ferrite magnets.

In some implementations, the magnet or magnets of a first magnetic implant may be made of a magnetic material that is not permanently magnetized, such as soft magnetic alloys, e.g., nickel-iron, silicon iron, iron, iron-cobalt, and ferritic stainless steels. In other words, the magnet(s) of respective magnetic implants may not be constructed of two permanent magnets. In other implementations, the magnets of a first and second magnetic implants may be constructed of two permanent magnets.

### Housing

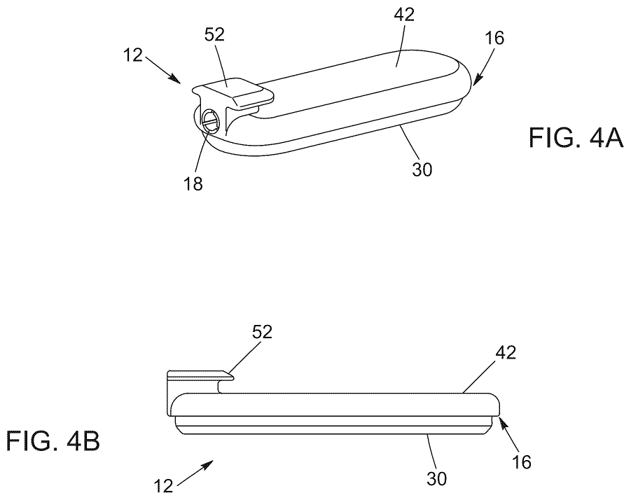

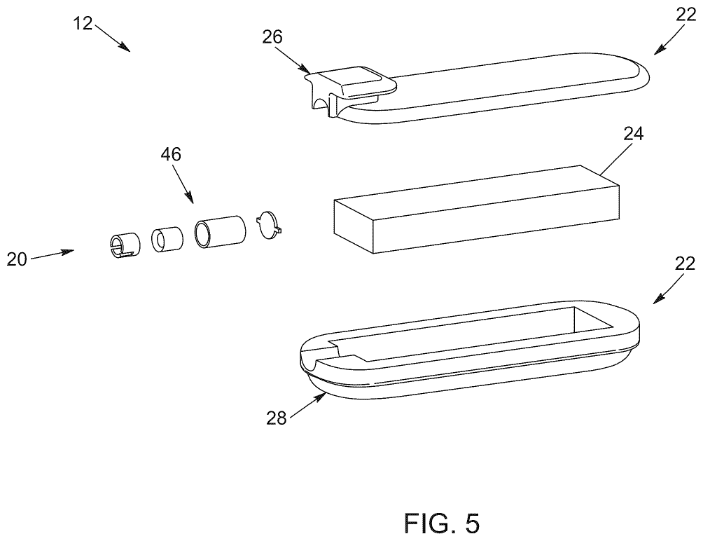

In some implementations, the magnetic implant 12, 14 can include a housing 22 configured to house a magnet therein. An example of housing 22 is shown in FIGS. 1 to 6. More particularly in FIGS. 4 and 5, the housing 22 is shown as including an outward portion 26 and an inward portion 28. In the context of the present description, the terms “outward” and “inward” when referring to the housing 22 are used in accordance with a radial reference system, in which the wall of the hollow organ is considered to be outwardly positioned relative to the lumen of the hollow organ. When the housing 22 is present, the outward portion 26 of the housing 22 is the portion that includes the lumen-oriented surface 42 of the magnetic implant, and the inward portion 28 of the housing 22 is the portion that includes the tissue-contacting surface or the compression surface 30. In the implementation shown, the outward portion 26 and the inward portion 28 together fully enclose a single magnet 24 therein. In other implementations, the single magnet can be fully enclosed in a single-piece housing, i.e., a housing 22 that is made of a single unit, the single unit including the tissue-contacting surface 30 and the lumen-oriented surface, 42 in accordance with the description above.

In some implementations, the magnetic implant 12, 14 can include a housing that is configured to receive multiple magnets therein. Providing multiple magnets within a single housing can contribute to enhancing the flexibility of the magnetic implant 12, 14, such that it can become easier to bend when subjected to a force. Alternatively, the multiple magnets can each be received in a corresponding housing, and the multiple magnets can be connected to each other by a cable, a string, a ribbon, a hitch, or a combination thereof. The description made above regarding the characteristics of the compression surface 30 of the magnetic implant is applicable to the housing 22 when the housing is present or when housings are present.

### Description of the Positioning Marker

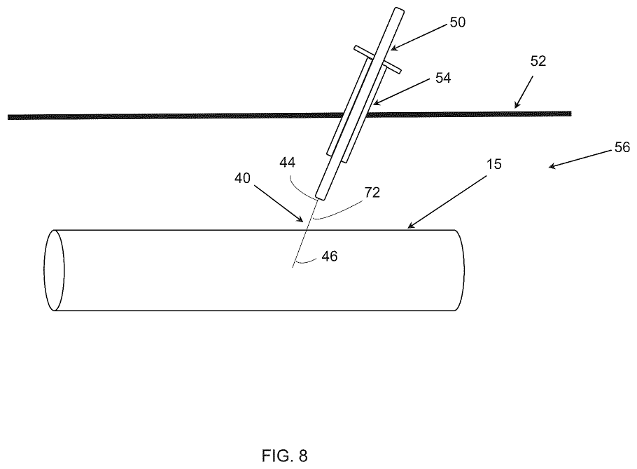

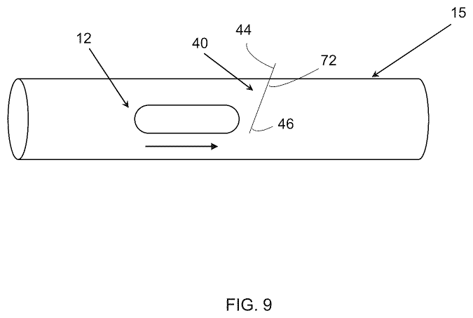

With reference now to FIGS. 8 and 9, an implementation of a positioning marker 40 is shown. In FIGS. 8 and 9, the positioning marker 40 is represented as an elongated member 72 that includes a proximal end 44 and a distal end 46. It is to be understood that the configuration, for instance in terms of shape and size, of the positioning marker 40 can vary, and that the elongated representation of the positioning maker 40 shown in FIGS. 8 and 9 is for exemplary purposes only.

The positioning marker 40 is configured to interact with a selected one of the first and second magnetic implants that is traveling in the digestive tract, for instance after being swallowed by a patient. For ease of reference, the selected one of the first and second magnetic implants will be referred to hereinbelow as “the magnetic implant”, which can be any chosen one of the first and second magnetic implants.

Swallowing the magnetic implant by the patient is an approach that can be taken to deliver the magnetic implant to the desired site of the anastomosis. In such implementations, the patient is given the magnetic implant to swallow, and the magnetic implant travels in the digestive tract via natural processes such as by peristalsis, similarly as food would do, for instance. Swallowing the magnetic implant and relying on natural processes such as peristalsis to have the magnetic implant travel down in the digestive tract can avoid an additional procedure involving the use of an endoscope by a health care provider, which in turn can ease the patient discomfort and increase the efficiency of the overall procedure. Based on expected travel times in the digestive tract, the healthcare provider can infer a potential location of the magnetic implant in the digestive tract after a predetermined period of time following swallowing.

For instance, it can be known from previous clinical studies that a magnetic implant having a given size and configuration can travel to a given location in the jejunum about 2.5 to 3.5 hours following swallowing. Based on this information, the healthcare provider can request the patient to swallow a first magnetic implant at a given time, wait for a certain period of time, prepare the delivery of a second magnetic implant, and then finalize the placement of the first and second magnetic implants so that the first and second magnetic implants can be magnetically coupled to each other. In other implementations, both the first and second magnetic implants can be swallowed by the patient, albeit at different times so that after a given period of time, the first magnetic implant has reached a first location on one side of a desired site of the anastomosis and the second magnetic implant has reached a second location on one side of the desired site of the anastomosis. This staggered approach can enable avoiding the use of an endoscope to deliver the first and second magnetic implants.

In order to ensure that the magnetic implant remains at a target location in the digestive tract that corresponds to the first location on one side of the desired site of the anastomosis, a positioning marker as described herein can be used. Given that the magnetic implant travels naturally down the digestive tract due to peristalsis, it is desired to avoid that the magnetic implant inadvertently travels past the target location. The positioning marker 40 is thus configured to intercept the magnetic implant as it travels down in the digestive tract so that the magnetic implant can remain at the target location.

The positioning marker 40 can be configured to be inserted into the lumen of the hollow organ of the digestive tract into which the magnetic implant is intended to be delivered. In order to do so, access into the abdominal cavity can be first gained using any suitable procedure known in the art, including minimally invasive surgeries. Minimally invasive surgeries can include laparoscopic surgeries, which typically includes cooperation of a laparoscopic instrument with a trocar to facilitate introduction of the laparoscopic instrument into the abdominal cavity, percutaneous laparoscopy, which can enable introduction into the abdominal cavity without the use of a trocar, and Natural Orifice Transluminal Endoscopic Surgery (NOTES) procedures, for example. The positioning marker can thus be couplable to a positioning marker guiding instrument that can be any type of instrument that can enable entering the abdominal cavity of the patient.

A NOTES procedure is a procedure that involves gaining access to the abdominal cavity by entering the digestive tract through a natural orifice rather than percutaneously. The natural orifice can vary depending on the location that is to be reached, and can include the mouth, the anus, or the vagina. Access via the mouth can enable a positioning marker guiding instrument to enter the stomach with the option of travelling further down in the digestive tract towards the small intestine, similarly to how an endoscope would be used, while access via the anus can enable a distal portion of the positioning marker guiding instrument to enter the colon and travel up towards the small intestine, similarly to how a colonoscope would be used. The wall of the digestive tract can then be breached to enable passage of the positioning marker guiding instrument therethrough such that the positioning marker guiding instrument can enter the abdominal cavity at a chosen location. When the NOTES procedure is performed via the mouth and the incision is made through the wall of the stomach, the procedure can be referred to as a transgastric NOTES procedure. The incision made through the wall of the digestive tract can then be sutured using endoscopically administered clips, for instance. A NOTES procedure can thus avoid an incision of the abdominal wall of the patient, which can also avoid complications that can occur from abdominal wall incisions, such as hernias or wound infections.

In some implementations, the positioning marker 40 can be a non-magnetic positioning marker, while in other implementations, the positioning marker 40 can be a magnetic positioning marker. When the positioning marker 40 is a non-magnetic positioning marker, the non-magnetic positioning marker can be configured to physically intercept the magnetic implant as it travels down in the digestive tract. When referring to a mechanical interception, it is intended to mean that the positioning marker can provide sufficient physical interference with respect to the magnetic implant to prevent the magnetic implant to travel further down in the digestive tract.

Referring to FIG. 8, the positioning marker 40 is shown as being coupled to a positioning marker guiding instrument, which is exemplified as a laparoscopic instrument 50. In other words, the proximal end 44 of the positioning marker 40 includes a positioning marker connector (not shown) configured to be coupled to the distal end of the laparoscopic instrument 50. The laparoscopic instrument 50 is configured to be introduced into the abdominal cavity 56 of the patient. In the implementation shown, the laparoscopic instrument 50 is inserted into the abdominal cavity 56 of the patient via a trocar 54 that is placed through the abdominal wall 52 of the patient.

Once in the abdominal cavity 56 of the patient, the distal end 46 of the positioning marker 40 can subsequently be inserted through the wall of the hollow organ where the target location for the magnetic implant is located. In the illustrated implementation, the hollow organ is exemplified as the jejunum, and thus the wall of the hollow organ is the jejunum wall 15. Given that in such implementations, the distal end 46 of the positioning marker 40 passes through the wall of the hollow organ, for instance by piercing, the distal end 46 of the positioning marker 40 can include, or be configured as, a pointed tip or a sharp tip. The portion of the positioning marker 40 that enters the lumen of the hollow organ also has a sufficiently small cross-section to enable closure of the opening once the positioning marker 40 is retrieved. Accordingly, in some implementations, the positioning maker 40, and more particularly the distal end 46 of the positioning marker 40, can be configured as a needle to facilitate passage through the wall of the hollow organ and entry into the lumen of the hollow organ.

In some implementations, the positioning marker 40, or a distal end 46 of the positioning marker 40 or another portion thereof, can include a floppy wire, or a floppy guidewire. A floppy wire is a flexible wire that can be made, for instance, of a spring coil. The presence of the floppy wire can advantageously provide flexibility to the positioning marker such that when the positioning marker intercepts the magnetic implant, the magnetic implant can still move slightly prior to being magnetically coupled to another magnetic implant, for instance due to the peristalsis that continues to be naturally applied on the magnetic implant. The floppy wire can include at least one segment that is magnetic and at least one segment that is non-magnetic. In some implementations, when the positioning marker 40 is configured as a floppy wire, or when the positioning marker 40 includes a floppy wire at a distal end 46 thereof, the magnetic segment of the floppy wire can be provided at a distal end of the floppy wire, and the non-magnetic segment can be provided at a proximal end of the floppy wire. In such implementations, the floppy wire thus includes a distal segment that is magnetic and a proximal segment that is non-magnetic, the distal segment being the one that can be magnetically attracted to the magnetic implant that the positioning marker is intended to intercept and thus that extends within the lumen of the hollow organ. At least a portion of the proximal segment that is non-magnetic can also extend within the lumen of the hollow organ, or an entirety of the proximal segment can also extend within the lumen of the hollow organ. The segment of the floppy wire that is magnetic can be made of any material that is magnetic and that can be magnetically coupled with the magnetic implant that the positioning marker is intended to intercept. For instance, the magnetic segment of the floppy wire can be made of a metal that is magnetically attracted to the magnetic implant, and the segment that is non-magnetic can be made of a polymer.

In some implementations, the distal end 46 of the positioning marker 40, or another portion thereof, can be configurable in a contracted configuration and a deployed configuration. For instance, the distal end 46 of the positioning marker 40 can include a deployable structure that is initially in a contracted configuration, with a smallest cross-section, to facilitate passage through the wall of the hollow organ and entry into the lumen of the hollow organ. Once into the lumen of the hollow organ, the deployable structure of the distal end 46 of the positioning marker 40 can adopt a deployed configuration in which the cross-section of the deployable structure increases, similarly to an umbrella-like structure for instance. Increasing the cross-section of the distal end 46 of the positioning marker 40 once the distal end 46 of the positioning marker 40 is within the lumen of the hollow organ can facilitate providing the desired interference with the magnetic implant to physically intercept the magnetic implant at the target location. For instance, the deployable structure can initially have a contracted configuration for passing through the wall of the hollow organ, then a deployed configuration once into the lumen of the hollow organ to physically intercept the magnetic implant, and once placement of the magnetic implant at the target location is achieved, the deployable structure can cycle back to the contracted configuration for retrieval from the lumen of the hollow organ.

In some implementations, the deployable structure can be made for instance of an expandable mesh that can adopt the deployed configuration and the contracted configuration as desired.

In some implementations, the deployable structure can include a balloon that, once within the lumen of the hollow organ, is expandable to reach a volume that can facilitate providing the desired physical interference with the magnetic implant to physically intercept the magnetic implant at the target location. Initially, the balloon can thus have a volume that facilitates delivery and placement of the positioning marker at the target location within the hollow organ, and then once the positioning marker has reached the target location, the volume of the balloon can be increased, i.e., expanded, to a volume that is sufficient for the balloon to provide the desired physical interference for intercepting the magnetic implant at the target location. In addition, in implementations where the deployable structure includes a balloon, the positioning maker can further include at a distal end thereof a magnetic tip that is configured to magnetically couple with the magnetic implant to further contribute to the interception of the magnetic implant, in addition to the physical interference provided by the balloon.

In other implementations, the positioning marker 40 can be configured as a T-fastener, or the deployable structure of the positioning marker 40 can include a T-fastener. When the positioning marker 40 is configured as a T-fastener, or includes a T-fastener, the positioning marker 40 can be configurable in a contracted configuration and a deployed configuration. In the contracted configuration, the distal end 46 of the positioning marker 40 is configured to extend longitudinally along the elongated member 72 of the positioning marker 40 to facilitate passage through the wall of the hollow organ and entry into the lumen of the hollow organ. Once the distal end 46 of the positioning marker 40, and optionally a portion of the elongated member 72, has entered the lumen of the hollow organ, the contracted configuration is switched to the deployed configuration, where the distal end 46 of the positioning marker 40 is deployed so as to extend along, or be substantially aligned with, the luminal wall surface of the hollow organ, e.g., transversally relative to the elongated member 72, rather than longitudinally along the elongated member 72. In some implementations, the distal end 46 can extend at an angle of about 90° relative to the elongated member 72, or between about 70° and 110° relative to the elongated member 72, for instance. In some implementations, the deployed configuration of the positioning marker 40, and the orientation of the distal end 46 that is different than longitudinally along the elongated member 72, can enable the distal end 46 of the positioning marker 40 to act as an anchor for the remaining of the positioning marker 40. In some implementations, a hollow needle can be used to pass through the abdominal wall 52 of the patient and the wall of the hollow organ, and enter into the lumen of the hollow organ to provide a passage for the insertion of the positioning marker 40. In such implementations, the elongated member 72 can be provided as a flexible wire, such as a suture or a polymeric or a metallic wire, and the distal end 46 can be made of a metal that can be magnetically attracted to the magnetic implant. Alternatively, the elongated member 72 can be substantially rigid, and can be made for instance of a metal or a rigid polymer, and the distal end 46 can be made of a metal that can be magnetically attracted to the magnetic implant.

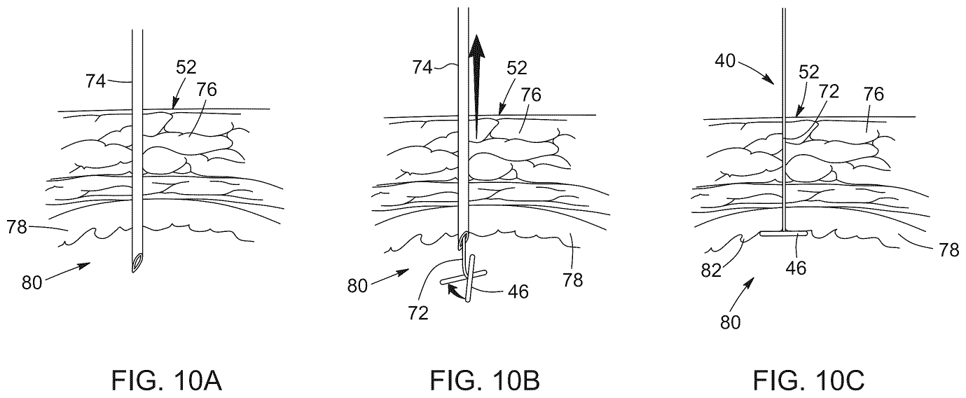

FIGS. 10A to 10C illustrate an implementation of a positioning maker 40 that is configurable in a contracted configuration and a deployed configuration. In FIG. 10A, a hollow needle 74 is shown inserted through layers 76 of the abdominal wall 52 and through the wall 78 of the hollow organ to reach the lumen 80 of the hollow organ. In FIG. 10B, the positioning marker 40 is inserted into the hollow needle, initially in a contracted configuration with the distal end 46 of the positioning marker 40 extending longitudinally along the elongated member 72, and then the positioning marker 40 travels downwardly enough such that the distal end 46 of the positioning marker 40 extends past the extremity of the hollow needle 74. Upon exiting the hollow needle 74, the distal end 46 of the positioning marker 40 is rotated, either automatically or manually, such that the positioning marker 40 adopts a deployed configuration, and the hollow needle is retrieved. In this implementation, the positioning marker 40 is configured as a T-fastener, and the combination of the elongated member 72 and the distal end 46 forms a shape that is similar to a “T” when the positioning marker 40 is in the deployed configuration. In FIG. 10C, the distal end 46 of the positioning marker 40 is shown abutted against the luminal wall surface 82 of the hollow organ, thus anchoring the positioning marker 40 at a target location along the digestive tract. Given that the distal end 46 can be made of a material that can be magnetically attracted to the magnetic implant, the distal end 46 of the positioning marker 40 can thus intercept the magnetic implant by magnetically coupling the positioning marker with the magnetic implant, at a target location along the digestive tract. It is to be understood that in other implementations, the distal end 46 of the positioning marker 40 can extend further away from the luminal wall surface 82 of the hollow organ, for instance as illustrated in the example shown in FIG. 9, such that the distal end 46 of the positioning marker 40 can intercept the magnetic implant at a target location along the digestive tract.

In some implementations, the positioning marker 40 can include a stabilizing feature (not shown) that can contribute to stabilizing the positioning marker once positioned at the target location. The stabilizing feature can be for instance a hook, a clip, such as a hemostatic clip (for instance, a Resolution™ clip), an endoscopic screw, an endoscopic corkscrew, or an endoscopic suture, that engages with the wall of the first hollow organ to maintain and stabilize the positioning marker at the target location. In some implementations, the clip can be radiopaque to facilitate its localization and visualization once implanted at the target location.

In some implementations, the stabilizing feature can also be any type of system that enables engagement of the positioning marker 40 with the wall of the hollow organ at the target location using various techniques. For instance, full thickness suturing of the positioning marker 40 can be performed to secure the positioning marker at the target location. Full thickness suturing incorporates the muscle layer in addition to the mucosa, which can contribute to efficiently secure the positioning marker 40 at the target location. The OverStitch™ endoscopic suturing system and the OverStitch Sx™ are examples of systems that can be used to perform full thickness suturing of the positioning marker 40. Alternatively, the positioning marker 40 can be engaged only with the mucosa of the hollow organ, at the target location. In some implementations, the stabilizing feature can include a knotless fixation, such as a Cinch™, to engage the positioning maker with the wall of the hollow organ without the need to tie a surgical knot.

In some implementations, the positioning marker 40 can include a defeatable portion. In the context of the present description, the term “defeatable” refers to the capacity of the defeatable portion to modify its configuration or structure after a predetermined period of time. The defeatable portion can be defeatable according to various mechanisms of which examples are provided in the following paragraphs.

The defeatable portion can be defeatable for instance mechanically or chemically via dissolution or degradation mechanisms.

When the defeatable portion is defeatable mechanically, it is meant that the defeatable portion has a structure that can be manipulated (e.g., by a person), for instance using an endoscope or externally, to directly or indirectly contact the defeatable portion to move the defeatable portion to a position or configuration that enables release from the engagement with the wall of the first hollow organ.

When the defeatable portion is defeatable via dissolution or degradation mechanisms, it is meant that the defeatable portion can be made of a material that is subjected to physical modifications of its configuration when in presence of selected conditions. For instance, the defeatable portion can be made a material that degrades sufficiently under the biological conditions present at the target location such that after a predetermined period of time, the degradation of the material is sufficient to release the engagement of the defeatable portion of the positioning marker with the wall of the first hollow organ. For instance, in implementations where the positioning marker 40 includes a stabilizing feature, the defeatable portion can be incorporated as part of the stabilizing feature. The defeatable portion can degrade as expected according to the biological conditions present at the target location, and after a predetermined period of time, be sufficiently degraded to enable release of the stabilizing feature from the engagement with the wall of the hollow organ. Other locations of the defeatable portion are also possible depending on the overall configuration of the positioning marker 40.

As mentioned above, the positioning marker 40 can be a non-magnetic positioning marker or a magnetic positioning marker. When the positioning marker 40 is a magnetic positioning marker, the magnetic positioning marker can be configured as described above for instance, and be made of a material that is magnetic to further contribute to the interception of the magnetic implant by magnetically coupling the positioning marker, or a portion thereof, with the magnetic implant upon the magnetic implant reaching the position marker, i.e., the target location, in the hollow organ. For instance, the distal end 46 of the positioning marker 40 can include a positioning marker magnet having a sufficient magnetic force to attract the magnetic implant as the magnetic implant travels down in the digestive tract. In such implementations, as the magnetic implant is approaching the positioning marker, the attracting force of the positioning marker magnet will guide and attract the magnetic implant to the target location so that the magnetic implant can remain at the target location rather than continuing traveling down in the digestive tract. The positioning marker magnet can thus have an opposite magnetic pole relative to a magnetic pole of the magnetic implant such that the magnetic implant can be attracted to the magnet of the positioning marker 40.

In some implementations, when the positioning marker 40 is a magnetic positioning marker as described above, the contribution of the positioning marker magnet, and thus the contribution of the attractive magnetic force conferred by the presence of the positioning marker magnet, to the interception of the magnetic implant can offer the possibility of designing the distal end 46 of the positioning marker 40 with a cross-section that can be reduced compared to if the positioning marker would not include a positioning marker magnet.

In some implementations, the positioning maker 40 can include one or more laparoscopic clips or endoscopic clips, which can each be magnetic or non-magnetic. The laparoscopic clip or endoscopic clip can be deployed at the target location in the digestive tract that corresponds to the intended location of the magnetic implant on one side of the desired site of the anastomosis. The laparoscopic clip can be positioned by the healthcare provider at the target location prior to the patient swallowing the magnetic implant, or after the patient has swallowed the magnetic implant. Similarly, the endoscopic clip can be positioned by the healthcare provider at the target location prior to the patient swallowing the magnetic implant, or after the patient has swallowed the magnetic implant. Once clipped at the target location, the laparoscopic clip or endoscopic clip can provide sufficient interference to physically intercept the magnetic implant travelling down in the digestive tract. The magnetic implant can then engage mechanically and/or magnetically with the clips, which can facilitate confirming that the magnetic has reached the target location. The laparoscopic clip or endoscopic clip can include any suitable feature that can facilitate the interaction with the magnetic implant and more particularly the interception of the magnetic implant as the magnetic implant travels down in the digestive tract.

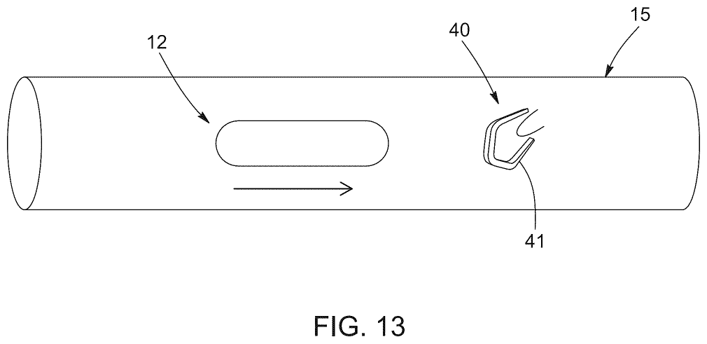

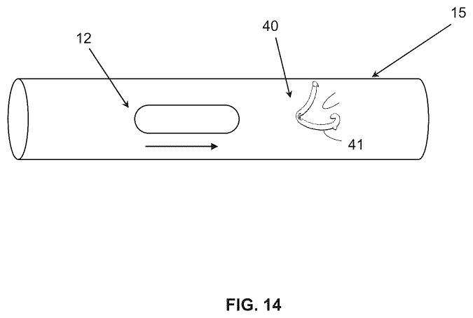

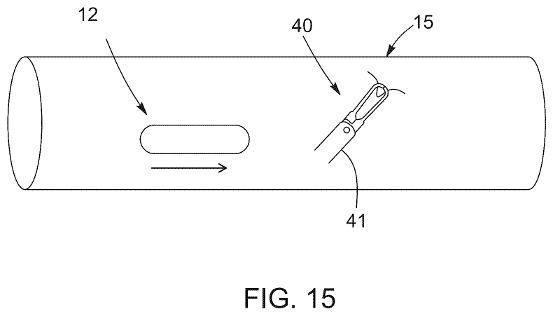

FIGS. 13 to 15 illustrate examples of a positioning marker 40 including an endoscopic clip 41 or a laparoscopic clip 41. The hollow organ is shown as being transparent, such that the illustrated clip 41 shown in FIGS. 13 to 15 can be interpreted as corresponding to an endoscopic clip clipped intraluminally or extraluminally to the wall of the hollow organ, or a laparoscopic clip clipped intraluminally or extraluminally to the wall of the hollow organ. In such implementations, the positioning marker 40 can thus be placed endoscopically or laparoscopically at the target location in the digestive tract that corresponds to the intended location of the magnetic implant on one side of the desired site of the anastomosis, and be engaged with the wall of the hollow organ either intraluminally or extraluminally. When the positioning marker 40 is engaged with the wall of the hollow organ extraluminally with the wall of the hollow organ, it is to be understood that the laparoscopic clip 41 can be configured to engage the magnetic implant magnetically, through the wall of the hollow organ. It is to be understood that other types of endoscopic clips and laparoscopic clips can be used than the illustrated examples.

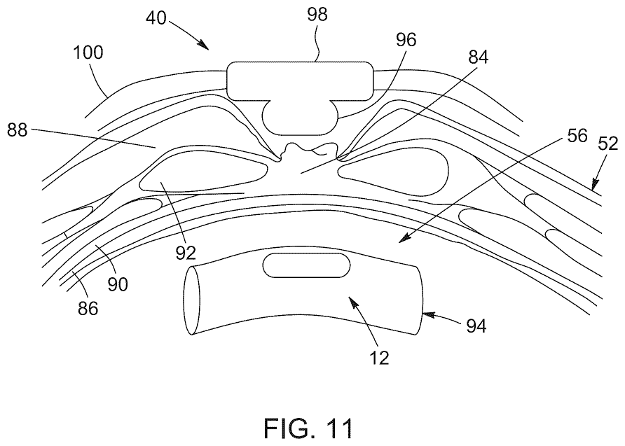

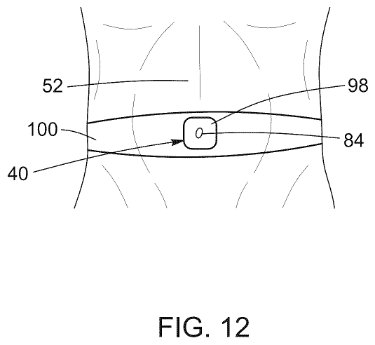

With reference to FIGS. 11 and 12, another implementation of a positioning marker 40 is shown. FIG. 11 illustrates a cross-section of the abdominal cavity 56, and the location of the organs that are present upon the umbilicus 84 (also referred to as the navel, or “belly button”), i.e., the peritoneum 86, the subcutaneous fat layer 88, the transversalis fascia 90 and the rectus abdominis muscle 92. In this implementation, the positioning marker 40 is configured to remain outside of the abdominal cavity of the patient. The positioning marker 40 is thus used externally of the patient, to facilitate the placement, or anchoring, of the magnetic implant 12 at a target location in the digestive tract, for instance after the magnetic implant 12, has been swallowed by the patient. The positioning marker 40 has a shape that is complimentary to the shape of the umbilicus 84. In the illustrated implementation, the positioning marker 40 is magnetic, and is configured to magnetically couple the magnetic implant 12 travelling in the digestive tract, exemplified as a portion of a small intestine 94 in FIG. 11. The magnetic coupling of the positioning marker 40 and the magnetic implant 12 enables the placement, or anchoring, of the magnetic implant 12 at a target location in the digestive tract.

In the implementation shown in FIGS. 11 and 12, the positioning marker 40 includes an umbilicus engaging portion 96 that is sized to fit into the umbilicus and optionally rest against the external surface of the umbilicus 84, and an abdominal wall abutting portion 98 configured to be placed onto the abdominal wall 52 of the patient, for instance to stabilize the umbilicus engaging portion 96 into the umbilicus 84. In some implementations, the umbilicus engaging portion 96 can thus have a substantially round or cylindrical shape, to better fit into the umbilicus 84. Although the abdominal wall abutting portion 98 is shown as having a substantially rectangular shape, the abdominal wall abutting portion 98 can also have a different shape, and for instance have a rectangular cross-section, an oval cross-section, or a circular cross-section.

In the implementation shown in FIGS. 11 and 12, the positioning marker 40 is combined with a belt 100, which can be formed of an elastic material or another type of material, to facilitate maintaining the positioning marker 40 in place and in particular, to maintain the placement of the umbilicus engaging portion 96 into the umbilicus. It is to be understood that only a portion of the belt 100 is shown in FIGS. 11 and 12, and that although not shown, the belt can encircle the abdomen of the patient as a “regular” belt would do. FIG. 12 illustrates a top view of the positioning marker 40, shown as being transparent to better illustrate the placement of the positioning marker 40 over the umbilicus 84. The size of the abdominal wall abutting portion 98 shown in FIG. 12 is for exemplary purposes only, and it is to be understood that the abdominal wall abutting portion 98, and more particularly the area delineated by the four peripheral walls of the abdominal wall abutting portion 98 (or the peripheral wall of the abdominal wall abutting portion 98 is the abdominal wall abutting portion 98 is oval or circular), can be larger or smaller than illustrated. Furthermore, although the belt 100 is shown as being wider than a width of the abdominal wall abutting portion 98, in other implementations, the width of the belt can be smaller than the width of the abdominal wall abutting portion 98.

In alternative implementations, a positioning marker can be incorporated in the magnetic implant. For instance, when the magnetic implant includes a housing configured to house a magnet therein, a fluorescent marker 43 or a radiopaque marker 45 can be incorporated into the housing or a portion of the housing. In such implementations, the positioning marker can enable optical or fluoroscopic visualization of the positioning of the magnetic implant by a health care provider following swallowing by the patient. Various types of fluorescent dyes can be used as a fluorescent marker 43, such as indocyanine green, CH1055, ethidium bromide, green fluorescent protein, rhodamine or fluoresceins, for instance. In some implementations, the positioning marker can take the form of a coating material that is fluorescent, the coating material being provided onto the magnetic implant, or onto the housing of the magnetic implant. For instance, the coating material can include a NIR-fluorescent dye such as Cy-C18 TPB, Cy-Bz TPB, Cy7.5, and Cy-Dp. In other implementations, the coating material can include a fluorescent dye such as phloxine B (PhB), eosin Y (Eos), indocyanine green (IcG) and calcein (Cal). It is to be understood that other dyes and types of dyes can be used, and that the above examples are given for exemplary purposes only. In some implementations, the coating material can be a lubricious coating provided on the magnetic implant, the lubricous coating including a fluorescein agent. Suitable medical instruments can then be used to detect the selected fluorescent marker 43 or a radiopaque marker 45.

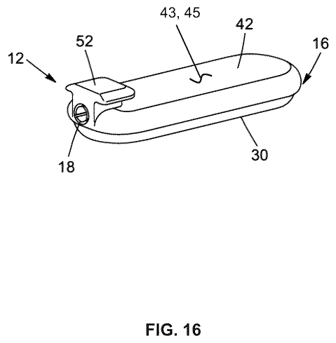

FIG. 16 illustrates an example of a positioning marker 43, 45 incorporated into the housing of a magnetic implant 12.

### Method for Forming an Anastomosis in the Digestive Tract

A method for forming an anastomosis between two adjacent walls of a digestive tract of a patient with first and second magnetic implants will now be described in further detail.

The method includes inserting the first magnetic implant into the digestive tract via a mouth of the patient so that the first magnetic implant can travel through the digestive tract and eventually reach a first hollow organ. Inserting the first magnetic implant into the digestive tract via the mouth of the patient can involve the patient swallowing the first magnetic implant as would typically be done for food. Natural processes associated with digestion can take place following the swallowing of the first magnetic implant, which can induce the travelling of the first magnetic implant down the digestive tract. The delivery of the first magnetic implant to the first hollow organ can thus rely on such natural processes, such as peristalsis, thereby alleviating the need for manipulating an endoscope to navigate the first magnetic implant through the digestive tract and deliver the first magnetic implant to the first hollow organ. In such implementations, the first magnetic implant can optionally be coated with a lubricious coating to aid in the swallowing. The patient may also have the opportunity to swallow the first magnetic implant while drinking a given volume of a liquid that can facilitate the swallowing, such as a liquid having an oil component. Alternatively or in addition, the throat of the patient may be contacted with a topical anesthetic to reduce discomfort when swallowing. It is to be understood that although the inserting of the first magnetic implant into the digestive system via the mouth of the patient can include swallowing the first magnetic implant, inserting of the first magnetic implant into the digestive system via the mouth of the patient can also involve the use of an endoscope, or another surgical instrument, if deemed appropriate by the healthcare provider, for instance in cases when a more rapid delivery of the first magnetic implant to the first hollow organ is desired.

Prior to or following the insertion of the first magnetic implant in the digestive tract of the patient, a positioning marker can be positioned at a target location corresponding to a first location for the first magnetic implant, on one side of a desired site of the anastomosis in the first hollow organ. In order to do so, the positioning maker can be initially releasably coupled to a positioning marker guiding instrument. The positioning marker guiding instrument can be inserted into the abdominal cavity of the patient via a minimally invasive procedure such as a laparoscopic procedure, an endoscopic procedure, or a NOTES procedure. When using a laparoscopic procedure or a NOTES procedure, the positioning marker guiding instrument can enter the abdominal cavity and can guide the positioning maker extraluminally of the digestive tract and thus of the first hollow organ. When using an endoscopic procedure, the positioning marker guiding instrument can enter the abdominal cavity and can guide the positioning maker intraluminally of the digestive tract and thus from within the digestive tract and the first hollow organ. Once the positioning marker reaches the target location, the positioning marker engages with the wall of the first hollow organ.

When the positioning marker is introduced into the abdominal cavity of the patient and guided extraluminally relative to the first hollow organ, the positioning of the positioning marker at the target location can include introducing at least a portion of the positioning marker into the lumen of the first hollow organ, thus across the wall of the first hollow organ. The at least a portion of the positioning marker can correspond to a distal end of the positioning marker, for instance. The distal end of the positioning marker can have a pointed tip or a sharp tip to facilitate insertion through the wall of the first hollow organ. Once the at least a portion of the positioning marker is inserted into the lumen of the first hollow organ, positioning the positioning marker at the target location can include stabilizing the positioning marker at the target location, for instance by clipping or hooking the positioning marker to the wall of the first hollow organ. Once it is determined that the positioning marker is sufficiently stabilized at the target location, whether via the use of a stabilizing feature or not, the positioning marker can be uncoupled from the positioning marker guiding instrument and the positioning marker guiding instrument can be retrieved from the abdominal cavity of the patient. Alternatively, the positioning marker and the positioning marker guiding instrument can remain coupled to each other until the first magnetic implant reaches the target location.

In implementations where at least a portion of the positioning marker is introduced into the lumen of the first hollow organ and the positioning marker includes a deployable structure, the positioning of the positioning marker at the target location can further include deploying the deployable structure to increase the cross-section of the distal end, or of another portion, of the positioning marker. The deployment of the deployable structure to increase the cross-section of the distal end of the positioning marker can also contribute to stabilizing the positioning marker at the target location. Accordingly, in such implementations, the presence of the deployable structure can be sufficient to stabilize the positioning marker at the target location, and the clipping or hooking the positioning marker to the wall of the first hollow organ can optionally be omitted.

When the positioning marker is introduced into the abdominal cavity of the patient and guided from within the lumen of the first hollow organ, i.e., intraluminally, the positioning of the positioning marker at the target location can include stabilizing the positioning marker at the target location, for instance by clipping or hooking the positioning marker to the wall of the first hollow organ. If it is determined that the positioning marker is sufficiently stabilized at the target location, whether a stabilizing feature is used or not, the positioning marker can be uncoupled from the positioning marker guiding instrument and the positioning marker guiding instrument can be retrieved from the abdominal cavity of the patient.

In implementations where the positioning marker is introduced into the abdominal cavity of the patient and guided from within the lumen of the first hollow organ, i.e., intraluminally, and the positioning marker includes a deployable structure, the positioning of the positioning marker at the target location can further include deploying the deployable structure to increase the cross-section of the distal end of the positioning marker. The deploying the deployable structure to increase the cross-section of the distal end of the positioning marker can also contribute to stabilizing the positioning marker at the target location. Depending on the configuration of the deployable structure, the presence of the deployable structure can be sufficient to stabilize the positioning marker at the target location, and the clipping or hooking the positioning marker to the wall of the first hollow organ can be present or be omitted.

Once the positioning marker is placed at the target location, the positioning marker will intercept the first magnetic implant on the one side of the desired site of the anastomosis. In some implementations, the interception of the first magnetic implant by the positioning marker can be the result of a mechanical interception, wherein the positioning marker can provide sufficient physical interference with the first magnetic implant to prevent the magnetic implant to travel further down in the digestive tract. In other implementations, the interception of the first magnetic implant by the positioning marker can result from the magnetic coupling of the first magnetic implant with a positioning marker magnet present in the positioning marker. In yet other implementations, the interception of the first magnetic implant by the positioning marker can result from both a mechanical interception of the first magnetic implant by the positioning marker and a magnetic coupling of the first magnetic implant with the positioning marker magnet.

The method also includes delivering the second magnetic implant to a second location on another side of the desired anastomose site. Various techniques can be used to navigate, or deliver, the second magnetic implant to the desired site of the anastomosis. It is to be noted that a chosen technique for navigating or deliver the first magnetic implant can be the same or different relative to the chosen technique for navigating or deliver the second magnetic implant. Thus, in some implementations, the second magnetic implant can be delivered to the second location on the other side of the desired anastomosis side using the same techniques as presented above regarding the delivery of the first magnetic implant to the target location. In such implementations, the patient may be asked to swallow the first magnetic implant at a given time, and then asked to swallow the second magnetic implant after a predetermined period of time, such that each one of the first and second magnetic implants can reach a first target location and a second target location, respectively, at moments that are suitable for the procedure. With such an approach, the second hollow organ and thus the second target location would be located downstream of the first hollow organ and thus of the first target location. In some implementations, in order for the second magnetic implant not to be intercepted at the first target location, the first positioning marker can be positioned subsequently to the second magnetic implant reaching the second target location.

In implementations where the second magnetic implant is delivered at the second location on the other side of the desired site of the anastomosis by a technique that is different than the techniques described above for delivering the first magnetic implant to the target location, i.e., at the first location, delivering the second magnetic implant can include releasably engaging the second magnetic implant with a delivery catheter insertable in a working channel of a corresponding endoscope via a connecting member, for instance. The endoscope (or colonoscope) can then be used to introduce the second magnetic implant into the digestive tract and to deliver the second magnetic implant at the second location.

In some implementations, the second magnetic implant can be navigated to the second location using a laparoscopic procedure. Details regarding various types of suitable laparoscopic procedures and laparoscopic instruments and devices can be found described in U.S. Patent Application No. 2020/0138438A1, which is incorporated herein by reference in its entirety.

Once the magnetic implants are delivered within their respective hollow organ and on their respective side the of the desired anastomosis, the first and second magnetic implants can be brought in close proximity to enable magnetic coupling of the first and second magnetic implants through the two adjacent vessel walls of the digestive tract, such that the compression surface of each of the first and second magnetic implants contacts the interior wall of their respective hollow organ at the site of the desired anastomosis.

Then, once the first and second magnetic implants are magnetically coupled to each other, the positioning marker located at the target location, i.e., at the first location, can be removed from the digestive tract and from the abdominal cavity of the patient. If a second positioning marker is used to intercept the second magnetic implant at the second location, the second positioning marker can also be removed from the digestive tract and from the abdominal cavity of the patient. Removing the positioning marker, and the second positioning marker if present, can be beneficial to not impair the magnetic coupling of the first and second magnetic implants.

Alternatively, depending on how the positioning member is stabilized and engaged with the wall of the hollow organ, the positioning marker can be excreted, or passed, naturally.

In implementations where the positioning marker includes a defeatable portion, the defeatable portion of the positioning marker can be defeated after a predetermined period of time to enable the positioning marker to be released from the target location and be excreted, or passed, naturally. The defeatable portion of the positioning marker can also be mechanically defeated, for instance using an endoscope.

The magnetic coupling of the two magnetic implants compresses a portion of the two adjacent walls therebetween, and the portion that is compressed between the respective compression surfaces of the magnetic implants eventually forms a necrotic area as the blood flood supply to this area progressively declines.

In some implementations, the first and second magnetic implants can be manipulated by using a magnet externally, for instance to facilitate the passing of the coupled magnetic implants via the bowel lumen of the patient once the healing time period is completed. An endoscope can also be used to manipulate the coupled magnetic implants internally, also to facilitate their passing via the bowel lumen of the patient once the healing time period is completed.

Another method for forming an anastomosis between two adjacent walls of a digestive tract of a patient with first and second magnetic implants will now be described in further detail.

According to this method, at least one of the first and second magnetic implants includes a fluorescent marker or a radiopaque marker.

The method comprises inserting the first magnetic implant into the digestive tract via a mouth of the patient so that the first magnetic implant travels through the digestive tract, as described above.

Then, a positioning of the at least one of the first and second magnetic implants can be detected as the at least one of the first and second magnetic implants travels through the digestive tract once swallowed by the patient, based on the fluorescent marker or a radiopaque marker.

The method can further include inserting a laparoscopic instrument in the abdominal cavity of the patient to position the at least one of the first and second magnetic implants at a target location corresponding to a first location on one side the desired site of the anastomosis. Examples of suitable laparoscopic procedures and laparoscopic instruments and devices can be found described in U.S. Patent Application No. 2020/0138438A1, which as mentioned above, is incorporated herein by reference in its entirety.

The method further includes delivering the second magnetic implant to a second location on another side of the desired anastomose site, with any technique deemed suitable. The chosen technique can be the same as the technique used to deliver the first magnetic implant to the first location, or can be different from the technique used to deliver the first magnetic implant to the first location, in accordance with the considerations presented above.

The first and second magnetic implants are then magnetically coupled the first and second magnetic implants to each other through the two adjacent vessel walls of the digestive tract to compress a portion of the two adjacent walls therebetween and form a necrotic area during a healing time period.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A system for forming an anastomosis between two adjacent walls of a digestive tract of a patient, the system comprising:
first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; and
a positioning marker positionable at a target location corresponding to a first location on one side the desired site of the anastomosis for the first magnetic implant, the positioning marker being configured to intercept the first magnetic implant at the target location once swallowed by the patient.

2. The system of claim 1, wherein the positioning marker is engageable with a positioning marker guiding instrument.

3. The system of claim 2, wherein the positioning marker guiding instrument comprises a laparoscopic instrument or an endoscopic instrument.

4. The system of claim 1, wherein the positioning marker comprises a floppy wire insertable through one of the two adjacent walls of the digestive tract.

5. The system of claim 4, wherein the floppy wire comprises a spring coil.

6. The system of claim 4, wherein the floppy wire comprises a distal segment that is magnetic, and a proximal segment that is non-magnetic.

7. The system of claim 1, wherein the positioning marker comprises a pointed tip at a distal end thereof to facilitate passage through one of the two adjacent walls of the digestive tract.

8. The system of claim 1, wherein the positioning marker comprises a needle at a distal end thereof to facilitate passage through one of the two adjacent walls of the digestive tract.

9. The system of claim 1, wherein the positioning marker is configurable in a contracted configuration for placement at the target location and a deployed configuration for intercepting the first magnetic implant at the target location.

10. The system of claim 9, wherein the positioning marker comprises a deployable structure.

11. The system of claim 10, wherein the deployable structure comprises a mesh, a T-fastener or a balloon.

12. The system of claim 1, wherein the positioning marker further comprises a stabilizing feature to stabilize the positioning marker once at the target location.

13. The system of claim 12, wherein the stabilizing feature comprises one or more of a clip and a hook.

14. The system of claim 12, wherein the stabilizing feature comprises an endoscopic suture.

15. The system of claim 1, wherein the positioning marker comprises a defeatable portion.

16. The system of claim 15, wherein the defeatable portion is configured to be defeatable mechanically using an endoscope.

17. The system of claim 15, wherein the defeatable portion is configured to be defeatable via a dissolution mechanism or a degradation mechanism.

18. The system of claim 1, wherein the positioning marker comprises a positioning marker magnet having an opposite magnetic pole relative to a magnetic pole of the first magnetic implant such that the first magnetic implant is attracted to the magnet of the positioning marker.

19. The system of claim 3, wherein the positioning marker comprises a laparoscopic clip.

20. The system of claim 19, wherein the laparoscopic clip comprises a positioning marker magnet having an opposite magnetic pole relative to a magnetic pole of the first magnetic implant such that the first magnetic implant is attracted to the magnet of the positioning marker.

21. The system of claim 3, wherein the positioning marker comprises an endoscopic clip.

22. The system of claim 21, wherein the endoscopic clip comprises a positioning marker magnet having an opposite magnetic pole relative to a magnetic pole of the first magnetic implant such that the first magnetic implant is attracted to the magnet of the positioning marker.

23. A system for forming an anastomosis between two adjacent walls of a digestive tract of a patient, the system comprising:
first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; at least one of the first and second magnetic implants comprising:
a fluorescent marker or a radiopaque marker to detect a positioning of the at least one of the first and second magnetic implants implant as the at least one of the first and second magnetic implants travels through the digestive tract once swallowed by the patient.

24. The system of claim 23, wherein the at least one of the first and second magnetic implants comprises a housing configured to house a magnet therein, and the fluorescent marker or the radiopaque marker is incorporated into the housing.

25. The system of claim 23, wherein the fluorescent marker comprises one or more of indocyanine green, CH1055 or a fluorescein.

26. A system for forming an anastomosis between two adjacent walls of a digestive tract of a patient, the system comprising:
first and second magnetic implants configured to magnetically couple to each other through the two adjacent walls of the digestive tract at a desired site of the anastomosis to compress a portion of the two adjacent walls therebetween and form a necrotic area that becomes surrounded by a scarred edge following a healing time period; and
a positioning marker comprising an umbilicus engaging portion positionable externally into an umbilicus of the patient, the positioning marker being configured to intercept one of the first and second magnetic implants at a target location once the magnetic implant is swallowed by the patient by magnetically coupling with the one of the first and second magnetic implants.

27. The system of claim 26, wherein the one of the first and second magnetic implants is the first magnetic implant, and the target location corresponds to a first location on one side the desired site of the anastomosis for the first magnetic implant.

28. The system of claim 26, wherein the positioning marker further comprises an abdominal wall abutting portion configured to be placed onto an abdominal wall of the patient to stabilize the umbilicus engaging portion into the umbilicus.

29. The system of claim 26, wherein the positioning marker further comprises a belt, the positioning marker being engaged with the belt and the belt being configured to further stabilize the positioning marker.
